(19) United States
(12) Patent Application Publication
(10) Pub. No.: US 2016/0145695 A1
(43) Pub. Date: May 26, 2016

(54) Epigenetic regulation of fruit trait with rapid periodic thermal shock treatment

(71) Applicant: Agrothermal Inc, Walnut Creek, CA (US)

(72) Inventors: Art Dawson, Chino Hills, CA (US); Martin Fischer, Milpitas, CA (US)

(21) Appl. No.: 14/797,655

(22) Filed: Jul. 13, 2015

Related U.S. Application Data
(60) Provisional application No. 61/998,977, filed on Jul. 14, 2014.

(51) Int. Cl. C12Q 1/68; A01G 1/00

(52) U.S. Cl. CPC C12Q 1/6895; A01G 1/001; C12Q 2600/154; C12Q 2600/13

(57) ABSTRACT

A method to improve fruit quality by manipulating the epigenetic trajectory of fruit in a commercially beneficial direction during the growth and ripening stages of the fruit by applying a special thermal stress treatment of the fruit in the field using periodic, rapid, short duration, pulses of heat. The levels of applied thermal stress are meant to keep the growth and ripening on the desired epigenomic trajectory. With such a process, targeted fruit traits may be consistently obtained.

### CROSS REFERENCE TO RELATED APPLICATIONS

This application claims priority from a U.S. Provisional Patent 61/998,977 filed on Jul. 14, 2014.

### FIELD OF THE INVENTION

The invention relates to the field of agriculture, more particularly to the field of molecular breeding. The present invention relates to a process for obtaining a targeted fruit quality parameter or a fruit trait in crop of fruit. The process in one part relates to a way to establishing specific epigenetic marker trajectories to obtain desired fruit qualities. In another part relates to implementing a thermal treatment device which causes the fruit to be developed along a desired epigenetic marker trajectory. And yet in another part relates to using the thermal treatment protocol to implement a specific epigenetic marker trajectory. In particular the epigenetic marker used is the DNA methylation state of the whole genome of the fruit cell.

### BACKGROUND OF THE INVENTION

The grape vine is one of the most abundant perennial crops in the world with a total surface of approximately 7.6 million hectares planted under vines. Its output, the grape berry, is an important commercial product. A significant portion of the output is dedicated to grapes cultivated for wine.

Maintenance and improvement of grape quality is a major research topic in agriculture. The need for understanding the science of grape berry growth/ripening and developing the tools to accomplish the same has many drivers. Beyond the obvious economic drivers, climate change considerations have gained an importance is recent years, driven by the extreme changes in the climate and the increasing variability in climate.

It is well known that environmental variables, such as light, water, temp, affect grape berry growth and ripening processes. Also known is that these environmental variables have an effect on the genetic machinery in developing grape berry cells. The grapevine vitis vinifera genome was developed in 2007. High-throughput sequencing technologies have been increasingly applied to gain a greater understanding of the regulation of physiological changes occurring during grape berry development. The emergence of tools such as microarrays, sequencing and next gen sequencing have been used in the past decade to understand the genetic and the epigenetic rationale in the ripening process with respect to the grape berry.

It is generally accepted that while the genetics machinery (genes in the genome) works to develop the traits of fruit, the regulating machinery of the epigenome serves to as control over the functioning of the genome. The epigenome responds to environmental stimuli and reprograms gene functions to adapt to the environmental stimuli.

In 2004¹ regulated-deficit irrigation was shown to improve berry and wine quality. Water availability, an environmental variable, if controlled can be used to control grape quality. ¹ Roby G, et al , Aust J Grape Wine Res 2004, 10:100-107

In 2009 Cramer et al² examined how water deficit alters differentially metabolic pathways affecting important flavor and quality traits in grape berries and that using new expression tools to explain certain specific impacts on specific ripening enzyme variations. In addition they³ also looked at transcriptomic and metabolomics profiles of grape berry development. ² Cramer et al BMC Genomics 2009, 10:212³ Cramer, Proc of the 2nd Annual National Viticulture Res Conf, Jul. 9-11, 2008, Univ of California, Davis

The emergence of drought as a result of climate change assumed increasing importance in research papers and studies show the impact of high temperature as a result of climate change on fruit quality have gathered momentum.

The use of modern tools of genetics are now brought in to explain the full impact of high and low temperature on fruit growth and fruit ripening. Genetic studies around transcript, Protein, enzyme, and metabolite profiling have been done for select fruits. Genetic explanations encompassing genes and transcriptional factors have been developed. However only select limited development has occurred in epigenome profiling of fruit development. Spotty epigenomic information exists on the grape berry. Below is detailed some of the relevant work by others in genetic and epigenetic understanding development with respect to grape berry and heat stress.

In November 2011 the first⁴ transcript and metabolite analysis in Trincadeira grapes revealed the dynamics of grape ripening. ⁴ Fortes et al, BMC Plant Biology, 2011, 11:149

In July 2011 Cohen⁵ established showed the impact of diurnal temperature cycle (effect of heat and light) on grape berry development and the flavonoid pathway genes. The exposure times for elevated temperatures were of the order of hours. ⁵ Cohen et al, Journal of Experimetnal Botany, Vol 63, No.7, pp 2655-2665, 2012

In February 2013 single base resolution of tomato fruit methylomes revealed epigenome modification associated with ripening⁶. It provides genome-wide insights into the link between the genetic program of fruit ripening and DNA methylation state. It identified DMRs (differentially methylated regions) and also showed that the epigenome is not static during fruit development. It suggests a potential for plant improvement strategies but does not provide any specific in that direction. Four stages of growth and ripening were examined. ⁶Zhong et al, Nature Biotechnology, Vol. 31 No. 2 FEBRUARY 2013

In February 2014, Reinth⁷ showed how grape berry could be impacted in a 24 hr. cycle. The study reveals that 2,684 transcripts of the 9,243 studied changed transcription during the day/night transition. It shows the effect of light on transcriptomic activity. In Apr 2014, Rienth ⁸ disclosed that day—night study on heat stress adaption of the grapevine berry shows that the transcriptome of fleshy fruits is differentially affected by heat stress at night. The paper showed transcriptomic differences as a result of application of heat ⁷ Rienth et al, PLoS ONE 9(2): e88844⁸ Rienth et al, BMC Plant Biology, 2014, 14:108 stress. The heat stress in this case is the continuous maintenance of elevated temperatures for hours at a time as opposed to a periodic short duration heat stress.

In June 2013 Osorio et al⁹ studied the molecular recognition of fruit ripening and acknowledged the role of epigenetic factors as potentially providing improved control over the ripening process. He suggested epigenetic based crop improvement strategies could radically impact fruit quality traits. However his recommendations were around understanding the epigenome so at better mitigate environmental variation. No specific strategies to control environment variables are described. ⁹ Osono et al, Frontiers in Plant Science, June 2013, Vol 4, Article 198

In July 2014 Agrothermal Inc. filed U.S. Provisional Patent 61/998,977 relating to the proactive use of special heat treatment profiles to impact epigenetic trajectories in fruit growth and fruit ripening and thereby maximize fruit quality traits.

Also in July 2014 a comparative study¹⁰ of ripening among berries of the grape cluster reveals an altered transcriptional program and enhanced ripening rate in delayed berries. While this indicated a distribution in transcriptomic events with respect to time, the final transcriptomic state of all the berries was the same at the end of the ripening period. This meant that differential expression exists within a bunch of berries. ¹⁰ S. Gouthu et al, Jour of Exp Botany doi:10.1093; Oregon Research Inst., OSU, Corvallis, Oreg., USA

In April 2015¹¹ Liu at al published a good review of genetic and epigenetic control of plants to heat stress. The review focuses on recent progresses regarding the genetic and epigenetic control of heat responses in plants, and pays more attention of the role of the major epigenetic mechanisms in plant heat responses. However this review while it studied heat stress effects on various species shows no work on vitis vinifera (See Table 1 in reference). Additionally the heat stress profiles in this reference differ considerably from those in the instant invention. The heat stress in this reference is the continuous maintenance of elevated temperatures for hours at a time as opposed to a periodic short duration heat stress as is the case in the instant invention. ¹¹ J, Feng L, Lii and He Z (2015) Genetic and epigenetic control of plant heat responses. Front. Plant Sci. 6:267.

In April 2015 Univ of Padova¹² indicated plans to study epigenetic control of ripening of the grape berry. Scientists plan to study histone modifications (H3K4me & H3k27me3) occurring in early and late stage of ripening of Cabernet Sauvignon berry grape. ChiP-Seq data will be integrated with transcriptomic data to see if the histone markers studied relate to genomic regions that include fruit ripening genes. ¹² Post Doc positon for research by University of Padova, Italy; attn.: Claudio Bonghi ; February 27, 2015

Recently in June 2015 Constantini et al¹³ published a detailed map of probable candidate genes for the fine regulation of color in grapes and the process to construct this map was well enumerated. A large amount of data was acquired and after bio-statistical analysis it formed a firmer basis for explaining the how the genetic machinery operates to determine grape color. While this mapping establishes the mechanistic genetic machinery of color determination, it does not address the role of epigenetic markers at the relevant gene loci to address the impact of an abiotic stress on grape color. On the other hand, the impact of abiotic stress on fruit trait is the subject of the instant invention. ¹³ Constantini et al, Journal of Experimental Botany Advanced Access, Jun. 12, 2015

While genetic explanation of impacts on flavonoid pathway and therefore grape quality have been presented, very little work has been done to detail the epigenetic control over the grape berry growth/ripening development. While all these studies present an improved scientific understanding of impact of heat on a plant's epigenetic machinery, there has been no suggestion of developing techniques to proactively implement such changes in directing the grape development along desired commercial paths. What is needed is method that shows how to calibrate desired grape berry development with epigenetic markers and a way to change those epigenetic markers in the field during actual berry development so as to result in a viable desired trait in the fruit grown.

It is notable in the literature that 1 hr. to 4 days of heat treatment at 37° C. has diverse effects on the epigenome suggesting the complexity of heat stress. The studies done to date are long exposures to a steady heat stress. Unlike all of the studies done to date, our inventive method studies the impact on the epigenome when subjected to a unique form of thermal stress- a rapid, short duration, periodic fashion.

### OBJECTIVE OF THE INVENTION

Improving fruit quality is an important commercial objective. The effect of climate change and the accompanying heat stress has been widely discussed. Thermal treatment to date has been used for pest control and frost control. However it has not been used to date to manipulate the epigenetic state of the fruit in the field. It is the objective of this invention to provide a method for such manipulation to better fruit quality.

### BRIEF SUMMARY OF INVENTION

What is proposed here is an inventive method to improve grape quality by first determining the optimal temperature modulation to achieve a desired specific grape berry quality parameter and then to subject grape berries in the field to this level of temperature modulation using TPT machines to result in a crop of targeted quality grape berries.

The optimal temperature modulation setpoints are obtained by correlating the degree of change in the epigenetic markers in the grape berry cell for various levels of heat stress. This is called epigenomic profiling. In particular, whole genome methylation epigenetic markers are characterized by determining the differentially Methylated Region (DMR) of the grape berry (TPT vs. control) using ChiP-seq and NextGen sequencing techniques.

### BRIEF DESCRIPTION OF DRAWINGS

FIG. 1. This figure shows a schematic representation of the temperature profile of a Rapid Periodic Thermal Stress applied to the fruit. It also shows the various parameters important for the characterization of the profile.

FIG. 2. This figure shows the process to define epigenetic control and comprises two definitive steps.

### DETAILED DESCRIPTION OF INVENTION

Our inventive process consists of two distinct process steps.

The first step determines the effective thermal stress to be applied to the grape berry to achieve certain fruit quality parameters and the second step applies such a step to the grape berry in the field to achieve the fruit quality desired. Completing both steps enables one to engineer desirable fruit qualities in the grape berry.

There are many fruit quality parameters of commercial interest. In the case of the grape berry one can use Brix, fruit set (berries/bunch), fruit yield (lbs./cluster, clusters/vine), anthocyanin content, as some of the fruit quality measures to be optimized. Such parameters can be individual values themselves or combinations of such parameters weighted appropriately to satisfy a commercial quality objective.

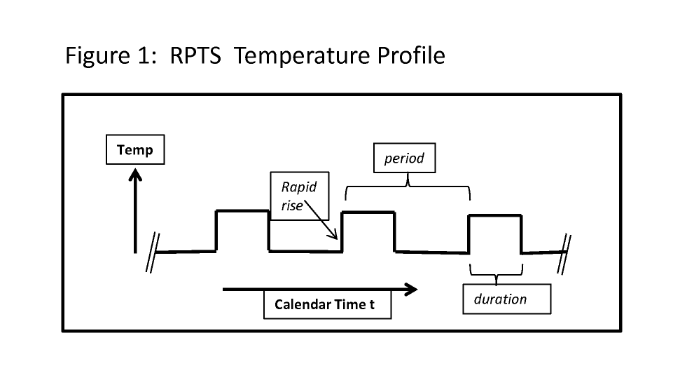

With RPTS (Rapid Periodic Thermal Stress) protocol on a TPT (Thermal Plant Treatment) machine, the thermal stress is characterized by the magnitude of temperature, duration and the periodicity. Any thermal stress can be mathematically represented by a square wave (or a saw tooth function) function over time where the amplitude represents the temperature. The duration of each thermal shock is the width of the square wave and the periodicity is defined by the time period between the square waves. See FIG. 1.

As part of the first step we perform an experimental design where various profiles of thermal stress are applied to the grape berry and the resulting changes in the methylation state of the whole genome of the grape berry are quantified.

Changes in methylation state can be studied by identifying and analyzing the Differentially Methylated Regions (DMR)in the whole genome of the grape berry using sequencing techniques. In plants, DNA methylation occurs at cytosine residues in three different sequences (CG, CHG, and CHH, where H=A, C or T. Usually genome promoter regions are hypomethylated and the remaining regions are hypermethylated. Analysis of epigenetic variation in Arabidopsis reveals that at least one-third of expressed genes are methylated in their coding region, and only 5% of genes are methylated within promoter regions. Changes to this normal pattern of methylation as a result of applying thermal stress is characterized in this step. DMR changes are usually measured as fold changes at different locations in the genome to aid rigorous statistical analysis.

Identification of DMR is done with the help of special tools. QDMR (Quantitative Differentially Methylated Regions) is a quantitative approach to quantify methylation difference and identify DMRs from genome-wide methylation profiles. This approach provides an effective tool for the high-throughput identification of the functional regions involved in epigenetic regulation. QDMR can be used as an effective tool for the quantification of methylation difference and identification of DMRs across multiple samples. Another tool to detect DMRs is Bioconductor's BiSeq which uses whole genome bisulfite sequencing data.

Analysis of DMRs that have been identified is also done with special software tools. methyAnalysis by Bioconductor is often used to visualize and analyze DNA methylation data. Yet another tool for analyzing DMRs is an open software package called Bsmooth.

The measured changes to the methylation state result in changes in fruit quality parameters. The changes in fruit quality parameters are mapped as a direct function of the methylation state of the genome. The methylation state of the genome is dependent on the thermal stress applied. Once this data quantification is completed, one is ready to go to the next step of the inventive process.

The second step of the invention is to apply the derived effective amount of thermal stress to the fruit so as to produce the desired fruit quality with the aid of selected level of stress derived from the information generated in the earlier first step.

The methylome of the grape berry changes over the growth and ripening cycle and methylome is typically characterized at selected specific points in the cycle.

Epigenetic regulation of the genome is achieved through several mechanisms. Amongst the known mechanisms today are DNA methylation, Post-translational histone modifications, histone variants, chromatin remodeling and with non-coding RNAs. In theory one can first map all of these on a genome wide basis so that the full regulatory effect of the genome can be understood. This task, in reality, has just begun. The complete science and the understanding in elucidating all of the epigenome programming is in its infancy. Such a complete task is a big task and the tools for doing so robustly and effectively are just emerging.

As a consequence and to serve as an example, we will only quantitatively characterize only one aspect of the epigenome—DNA methylation of the whole genome in the instant invention. DNA methylation characterization techniques are fairly well developed. However, this should not be construed as a limitation. The other remaining epigenetic mechanisms may turn out to provide an even finer resolution in mapping of thermal stress effects on the fruit epigenome.

Modulated heat stress has been shown to increase grape berry quality parameters in many studies done at Agrothermal Inc. The heat stress is applied to the grape berry by a procedure known as Thermal Plant Treatment (TPT) or Rapid Periodic Temp Shock Treatment (RPTS) with a thermal profile as shown in FIG. 1. The modulation is controlled both in terms of temperature, duration and periodicity and is usually applied in specific calendar time periods of grape berry growth and ripening cycle. The effect of this treatment is that the plant is routinely stressed with an abiotic heat stress over a period of time. This alters its epigenetic programming in a commercially favorable direction.

The inventors have separately invented a machine and a process for applying heat stress to fruits in the field. The machine is called a TPT machine (Temperature Plant Treatment) and is described in an earlier U.S. patent application Ser. No. 13/261,934 by the same inventors and is incorporated herein by reference. Without going in to the equipment details all described in the referenced application, it would suffice to say that a TPT machine is now being used today to apply a modulated level of heat stress to fruits in the field.

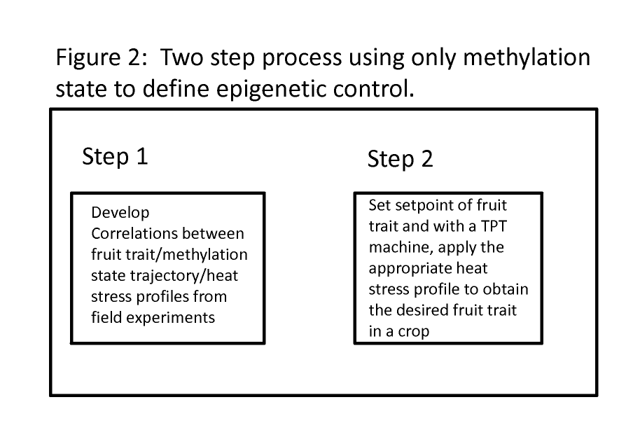

FIG. 2 describes the two step process in a graphic manner. At first methylation state trajectory impact of a known temperature profile is assessed. The resulting fruit quality trait is then correlated with the achieved methylation trajectory. Many field experiments are needed to derive this information and appropriate correlations for various profiles of applied heat stress. Armed with this information, one can then apply a specific temperature profile to the fruit to get the correlated methylation trajectory and the concomitant level of a fruit trait.

With such a powerful ability to target fruit quality as evidenced by this inventive process, it is likely that such process could go beyond crop improvement strategy. These methods and processes could be adopted by nutraceutical manufacturers to maximize nutritive biochemical content in their fruit supply in the field prior to extraction. As an example, it would be possible to maximize anthocyanin or reservatrol content using such a process and be able to supply raw materials to the nutraceutical industry.

### GLOSSARY OF TERMS USED

Epigenome marker—the specific chemical ‘punctuation’ on the genome that alters the function of the genome. This can be accomplished by the various mechanisms mentioned earlier and known in the literature .e.g. DNA methylation, histone tail modifications, etc.

Epigenetically effective—sufficient to result in a certain state of epigenome markers on the genome of the fruit.

Epigenetic state—the epigenome configuration as defined by a specific combination of epigenetic markers; also called epigenetic programming.

Periodic stress—stress that is applied at different times. Eg. once a day, twice per week, once a month, etc.

Rapid stress—stress that achieves its application level very quickly.

Duration of stress—the period of time the stress is held at its application level.

Abiotic stress—stress from environmental inputs like UV, visible light , water, heat.

Epigenome trajectory—the epigenome is not static during the growth or ripening process. It changes and has a certain trajectory in time.

Modulated level of stress—a controlled level of stress.

Heat stress and thermal treatment—They are used interchangeably in this application. Both mean the use of heat.

Epigenomic profiling—characterization of epigenetic markers for a given genome.

TPT—Thermal Plant Treatment

TPT Machine—Equipment that applies TPT to plants in the field.

RPTS—Rapid Periodic Thermal Shock Treatment. It is a subset of TPT treatment and specifies the nature of the heat exposure in time.

Fruit Quality Parameter—a trait usually of commercial value; beneficial parameter; it is a phenotypic expression; visual marker as they can be visualized; interchangeably used with fruit trait.

Trait Loci—locations of the genes in the entire genome that contribute to a particular fruit trait

We claim:

1. A process to improve targeted fruit trait comprising the steps of:
a) determining the effective amount of change in epigenetic regulation necessary to achieve targeted trait comprising the steps of:
1) establishing quantitative trait loci for the fruit,
2) exposing fruit to a specific level of abiotic stress,
3) extracting the whole genome of the fruit from the fruit cells,
4) determining the epigenetic profile of the whole genome using gene sequencing tools,
5) determining the change in epigenetic regulation profile at the relevant trait loci from that in a control sample,
6) repeating steps 2) thru 5) at various levels of applied abiotic stress,
7) correlating the change in epigenetic profile with the applied level of abiotic stress,
8) determining the epigenetically effective modulated level of abiotic stress to attain the targeted level of trait,
b) and treating the fruit in the field with the modulated level of abiotic stress for an epigenetically effective period of time.

2. The process of claim 1 wherein the epigenetic profile is defined by the operation of epigenetic mechanisms from the group consisting of DNA methylation, post translational histone modifications, chromatin remodeling, histone variants and non-coding RNA or combinations thereof.

3. The process of claim 1 wherein the epigenetic profile is entirely defined by DNA methylation.

4. The process of claim 1 wherein the said abiotic stress is heat stress.

5. The process of claim 1 wherein the fruit is a grape berry.

6. The process of claim 4 wherein the heat stress applied to the fruit is applied rapidly, has a short duration of less than 20 seconds and is applied periodically over time.

7. The process of claim 4 where heat stress is applied by a TPT machine.
